(19) United States
(12) Patent Application Publication
(10) Pub. No.: US 2016/0194126 A1
(43) Pub. Date: Jul. 7, 2016

(54) ENCLOSABLE CONTAINER SYSTEM AND SEALING INDICATOR

(71) Applicant: Brian R. FINDLAY, Pepperell, MA (US)

(72) Inventor: Brian R. FINDLAY, Pepperell, MA (US)

(21) Appl. No.: 14/969,134

(22) Filed: Dec. 15, 2015

Related U.S. Application Data
(60) Provisional application No. 62/099,187, filed on Jan. 1, 2015.
(60) Provisional application No. 62/104,962, filed on Jan. 19, 2015.
(60) Provisional application No. 62/132,000, filed on Mar. 12, 2015.

(51) Int. Cl. B65D 55/00

(52) U.S. Cl. CPC B65D 55/00

(57) ABSTRACT

A container system comprising a cover and an enclosable body. The cover includes a central region, at least one sidewall extending from the central region, a first mating interface, and a first sealing interface. The enclosable body comprises at least one sidewall defining a cavity having an opening, a second mating interface disposed on the at least one sidewall, the second mating interface engaging the first mating interface, a second sealing interface disposed on the at least one sidewall, the second mating interface engaging the first sealing interface to form a seal therebetween, and a sealing indicator disposed within on the at least one sidewall, the sealing indicator configured to indicate that the cover is sealed to the enclosable body.

### CLAIM OF PRIORITY

The present application claims the benefit of provisional application No. 62/099,187, filed on Jan. 1, 2015, provisional application No. 62/104,962, filed on Jan. 19, 2015, and provisional application No. 62/132,000, filed on Mar. 12, 2015, each of which are fully incorporated herein by reference.

### TECHNICAL FIELD

The present disclosure relates to containers and more specifically to enclosable containers.

### BACKGROUND INFORMATION

There are many applications where it is appropriate to enclose an open container. For example, various beverage cups, cartons for holding physical objects, and even syringes. Enclosure methods are generally limited to a press fit, a friction fit, or a threaded connector. An inherent limitation to these methods is determining whether the requisite amount of force has been applied to the respective method of enclosing the container in order to properly close the container. Prior solutions have involved utilizing an audible noise that is emitted when the container is properly closed or have employed a physical locking system. Clearly these solutions are not ideal, as each suffers from unnecessary complexity and questionable functionality. The present disclosure seeks to remedy these problems.

### SUMMARY

An embodiment of an enclosable body consistent with this disclosure includes at least one body-sidewall defining a cavity having an opening. A first mating interface is disposed on the at least one body-sidewall substantially adjacent to the opening. A first sealing interface is disposed on the at least one body-sidewall. An enclosed indicator is disposed within the first mating interface.

An embodiment of a covering element consistent with this disclosure includes at least one covering element having at least one covering-sidewall and a base. A first mating interface is disposed on the at least one covering-sidewall. A first sealing interface is disposed on at least one of the covering-sidewall or the base. At least one inspection element is disposed in the at least one covering-sidewall for revealing an enclosed indicator.

An embodiment of a container consistent with this disclosure includes at least one body-sidewall defining a cavity having an opening. A first mating interface is disposed on the at least one body-sidewall substantially adjacent to the opening. A first sealing interface is disposed on the at least one body-sidewall. An enclosed indicator disposed within the first mating interface. A covering element having at least one covering-sidewall and a base, a second mating interface is disposed on the at least one covering-sidewall, the second mating interface engages the first mating interface, and a second sealing interface is disposed on at least one of the covering-sidewall or the base.

### BRIEF DESCRIPTION OF THE DRAWINGS

These and other features and advantages will be better understood by reading the following detailed description, taken together with the drawings, wherein:

FIG. 1 depicts a diagrammatic view of an enclosable body and a cover/lid including a sealing indicator in an unsealed state.

FIG. 2a depicts a cross-sectional view of an enclosable body and a cover/lid including a sealed friction connection.

FIG. 2b depicts a cross-sectional view of the enclosable body and cover/lid of FIG. 2a unsealed.

FIG. 3a depicts another cross-sectional view of an enclosable body and a cover/lid including a sealed friction connection.

FIG. 3b depicts a cross-sectional view of the enclosable body and cover/lid of FIG. 3a unsealed.

FIG. 4a depicts another cross-sectional view of an enclosable body and a cover/lid including a snap fit connection.

FIG. 4b depicts a cross-sectional view of the enclosable body and cover/lid of FIG. 4a unsealed.

FIG. 4c depicts an enlarged view of the snap fit of FIG. 4a sealed.

FIG. 4d depicts an enlarged view of the snap fit of FIG. 4a unsealed.

FIG. 4e depicts an enlarged view of another embodiment showing a sealed snap fit connection and a sealing indicator.

FIG. 5 depicts a diagrammatic view of an enclosable body having a hashed sealing indicator.

FIG. 6 depicts a diagrammatic view of an enclosable body having a gradation sealing indicator.

FIG. 7 depicts a diagrammatic view of an enclosable body having a jagged sealing indicator.

FIG. 8 depicts a diagrammatic view of an enclosable body having a square wave sealing indicator.

FIG. 9 depicts a diagrammatic view of an enclosable body having a dashed line sealing indicator.

FIG. 10 depicts a diagrammatic view of an enclosable body including a sealing indicator and a cover/lid including at least one inspection element unsealed.

FIG. 11 depicts the enclosable body and cover/lid of FIG. 10 sealed.

FIG. 12 depicts a diagrammatic view of another embodiment of an enclosable body including a sealing indicator and a cover/lid including at least one inspection element unsealed.

FIG. 13 depicts the enclosable body and cover/lid of FIG. 12 sealed.

FIG. 14 depicts a diagrammatic view of yet another embodiment of an enclosable body including an unsealed indicator and a cover/lid including at least one inspection element unsealed.

FIG. 15 depicts the enclosable body and cover/lid of FIG. 14 sealed.

FIG. 16 depicts a diagrammatic view of an enclosable body having a covering element that is not fully engaging the enclosable body.

FIG. 17 depicts a diagrammatic view of syringe using a sealing indicator.

FIG. 18 depicts a diagrammatic view of a box having a sealing indicator.

FIG. 19 depicts a diagrammatic view of a pipe system having a male and female pipe wherein the male pipe has a sealing indicator.

FIG. 20 depicts a diagrammatic view of a pipe system having two male pipes to be joined using a coupler.

### DETAILED DESCRIPTION

By way of a general overview, a sealable container consistent with at least one embodiment described herein generally includes an enclosable body defining an open cavity, a cover/lid, and at least one sealing indicator. The sealing indicator is configured to provide a quick and easy mechanism for a user and/or machine to verify that the cover/lid has been sealed to the enclosable body.

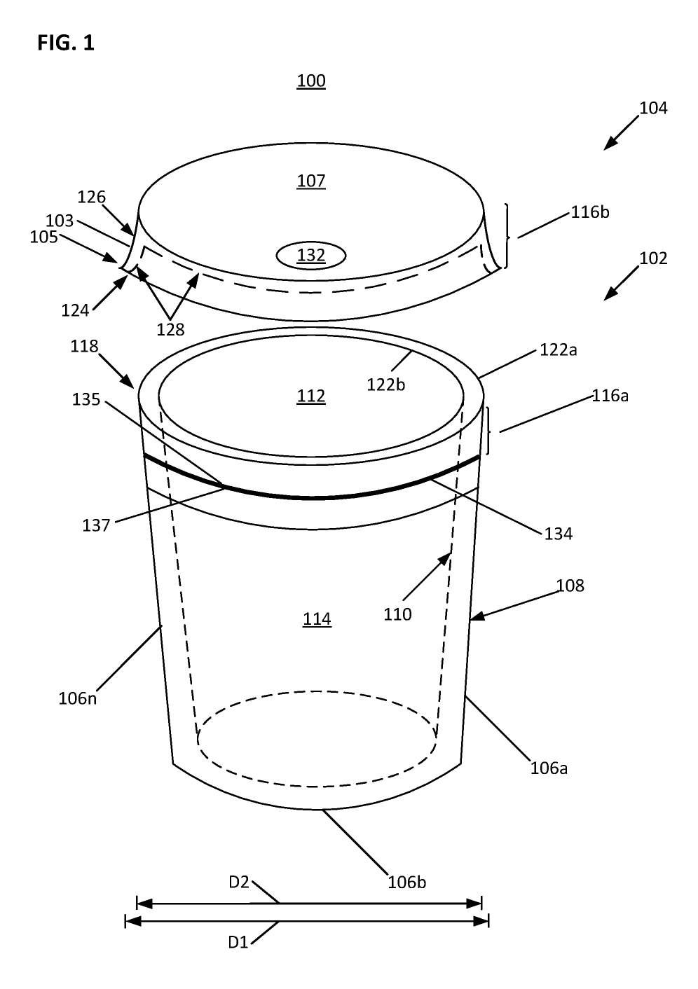

Referring to FIG. 1, a sealable container 100 including an enclosable body 102 and a cover/lid 104 is generally depicted. Non-limiting examples of the enclosable body 102, consistent with the present disclosure, may include a cup, a box, a syringe, an ice cream container, a bowl, or any other object capable of retaining liquids, solids, and/or gases. The enclosable body 102 may include at least one sidewall and/or base 106a-n (collectively 106), each of which may have an outer surface 108, an inner surface 110, and an opening 112. The inner surface 110 and the opening 112 define at least one cavity 114 for retaining any combination of solids, liquids, and/or gases.

The cover/lid 104 may include one or more sidewalls 103 extending generally downward from one or more upper, top, and/or central regions 107. The cover/lid 104 may optionally include one or more openings/apertures 132 for dispensing material contained within the cavity 114. Additionally (or alternatively), the sidewalls 103 may optionally include one or more lips or flanges 105 extending generally radially outward from a distal end of the sidewall 103. The lip or flange 105 may have an interior dimension D1 (e.g., circumference) that is larger than the external dimension D2 (e.g., circumference) of the sidewall 106n proximate to the opening 112. As may be appreciated, the larger internal dimension D1 of the lip or flange 105 may facilitate placing/aligning the cover/lid 104 over/around the opening 112 of the enclosable body 102. It should be appreciated that a portion of the sidewall 103 (e.g., but not limited to, the lip/flange 105) may not directly contact the outer surface 108 of the enclosable body 102 when the cover/lid 104 is sealed.

The enclosable body 102 may include one or more mating interfaces 116a and/or sealing interfaces 118 to generally form one or more connections and seals between the enclosable body 102 and the cover/lid 104. As may be appreciated, in some embodiments, one or more of the mating interfaces 116a may include one or more of the sealing interfaces 118, but in other embodiments one or more of the mating interface 116a may be separate and distinct from one or more of the sealing interface 118a. Further, in some embodiments, the seal may be temporary (for example, the user may selectively create the seal by placing the cover/lid 104 on, or removing the cover/lid 104 from, the enclosable body 102) or the seal may be different for different contents (the seal may be, for example, but not limited to, any one of vapor-tight, liquid-tight, hermetic, and/or capable of only retaining solids).

The mating interface(s) 116a of the enclosable body 102 generally correspond to one or more regions of the outer surface 108 and/or the inner surface 110 of the sidewall 106 which engage one or more corresponding mating interfaces 116b of the outer and/or inner surfaces 126, 128 of the cover/lid 104 to couple, mount, secure, and/or generally retain the cover/lid 104 to the enclosable body 102. As used herein, the cover/lid 104 is considered to be generally mounted, secured, and/or retained to the enclosable body 102 when a connection is formed therebetween which is sufficient to keep the cover/lid 104 engaged to the enclosable body 102 during normal and/or intended use of the sealable container 100. For example, in an embodiment in which the enclosable body 102 and the cover/lid 104 are a cup and a lid for holding a liquid beverage, the cover/lid 104 and the enclosable body 102 are considered to be generally mounted, secured, and/or retained together when the cover/lid 104 remains engaged to the enclosable body 102 when dispensing the liquid therefrom (e.g., during drinking and/or pouring) such that the cover/lid 104 does not separate from the enclosable body 102. As may be appreciated, in some embodiments the cover/lid 104 may be generally mounted, secured, and/or retained to the enclosable body 102 irrespective of a seal being formed between the cover/lid 104 and the enclosable body 102 as discussed herein. In some embodiments, the mating interfaces 116a, 116b may include, but are not limited to, a friction fit/connection, an interference fit/connection, mechanical fit/connection, or a snap fit/connection. By way of a non-limiting example, the mating interface(s) 116a, 116b may be located substantially adjacent to an edge 122a, 122b and/or proximate to the opening 112.

For example, the friction fit and/or interference fit may be formed between the inner surface 128 of the cover/lid 104 and the outer surface 108 of the enclosable body 102 and/or the outer surface 126 of the cover/lid 104 and the inner surface 110 of the enclosable body 102. In some embodiments, the friction fit and/or interference fit may be formed by forcibly expanding (or inserting) the cover/lid 104 over the outer surface 108 of the enclosable body 102 or into the inner surface 110 of the cavity 114. As should be appreciated, in some embodiments the enclosable body 102 may be forcibly expanded over (or inserted into) the cover/lid 104. In these embodiments, one or both of the cover/lid 104 and the enclosable body 102 may be made, at least partially, of a resiliently deformable material (e.g., plastics, silicones, elastomers, and etc . . . ).

The sealing interface(s) 118 of the enclosable body 102 may interact with one or more corresponding sealing interface(s) 124 of the outer and/or inner surfaces 126, 128 of the cover/lid 104 to form a seal between the enclosable body 102 and the cover/lid 104. As used herein, a seal is formed between the enclosable body 102 and the cover/lid 104 when the cover/lid 104 is generally mounted, secured, and/or retained to the enclosable body 102 in such a manner to generally prevent leakage of a material disposed within the cavity 114 at the sealing interfaces 118, 124 during normal and/or intended use of the sealable container 100. For example, in an embodiment in which the enclosable body 102 and the cover/lid 104 are a cup and a lid for holding a liquid beverage, the cover/lid 104 and the enclosable body 102 are considered be sealed when the sealing interfaces 118, 124 generally prevent leakage between the enclosable body 102 and the cover/lid 104 of the material in the cavity 114 (e.g., at the sealing interfaces 118, 124) during use of the sealable container 100 (e.g., when dispensing and/or drinking from the sealable container 100). The phrase “generally prevent leakage” is intended to mean that the leakage between the enclosable body 102 and the cover/lid 104 (e.g., at the sealing interfaces 118, 124) of the material in the cavity 114 is eliminated and/or reduced to an acceptable rate based on the intended use of the sealable container 100. The acceptable rate of leakage may be defined as an amount of leakage that is acceptable to the user, manufacturer, and/or distributor (e.g., retail store) of the sealable container 100. For example, an acceptable rate of leakage may be less than or equal to 10% (such, as, but not limited to, less than or equal to 5% or less than or equal to 2%) of flow rate through the opening 132 in the cover/lid 104 while dispensing material from the cavity 114 (e.g., drinking). The effectiveness of the sealing interfaces 118, 124 may include and/or be enhanced through use of, for example, one or more optional gaskets and/or O-rings.

The exact location(s) of the sealing interfaces 118, 124 will depend on the specific application, though one or more of the sealing interface(s) 118 may be formed using all or part of the outer surface(s) 108 and/or the inner surface(s) 110 of the enclosable body 102 and one or more of the sealing interface(s) 124 may be formed using all or part of the outer surface(s) 126 and/or the inner surface(s) 128 of the cover/lid 104. For example, when one or more sealing interfaces 124 are formed on the outer surface(s) 126 of the cover/lid 104, one or more corresponding sealing interfaces 118 may be located on the inner surface(s) 110 (such, as, but not limited to, edge 122b) of the enclosable body 102. Alternatively (or in addition), when one or more sealing interfaces 124 are formed on the inner surface(s) 128 of the cover/lid 104, one or more corresponding sealing interfaces 118 may be located on the outer surface 108 (such as, but not limited to, edge 122a) of the enclosable body 102.

The sealable container 100 may also include one or more sealing indicators 134 configured to allow a user and/or a machine to easily and quickly verify that the cover 104 is sealed to the enclosable body 102. As explained herein, the sealing indicator 134 may be located on a portion of the outer and/or inner surfaces 108, 110 of the enclosable body 102 such that at least one of a top and/or bottom edges 135, 137 is obscured by a portion of the cover 104 only when the cover 104 is sealed to the enclosable body 102. For example, the sealing indicator 134 may be located on a portion of the outer and/or inner surfaces 108, 110 of the enclosable body 102 such that at least one of a top and/or bottom edges 135, 137 is obscured by a portion of the sidewall 103 and/or flange 105 of the cover 104 only when the cover 104 is sealed to the enclosable body 102. As such, a user and/or machine may quickly and easily verify that the cover 104 is sealed to the enclosable body 102 when at least one of the top and/or bottom edges 135, 137 is obscured by a portion of the cover 104. Put another way, the placement of the sealing indicator 134 is selected such that at least one of the top and/or bottom edges 135, 137 is obscured by a portion of the cover 104 only when the cover 104 is sealed to the enclosable body 102.

As used herein, the sealing indicator 134 is considered to be obscured when the sealing indicator 134 is concealed, covered, masked, and/or blocked when analyzed from a viewing angle V which is defined as an angle extending from a plane P coextensive with the selected edge 135, 137 used for verification, and which extends generally towards the cover/lid 104 when the cover/lid 104 engages with (e.g., seals) the enclosable body 102.

As described herein, the sealing indicator 134 may take many different forms. It should be appreciated that at least one of the top or bottom edge 135, 137 of the sealing indicator 134 is different than any surrounding indicia on the enclosable body 102. Put another way, at least one of the top or bottom edge 135, 137 of the sealing indicator 137 is sufficient unique/different from the portions of the enclosable body 102 that are adjacent to the edge 135, 137 to allow a user and/or machine to differentiate the edge 135, 137 of the sealing indicator 134 from the adjacent portions of the enclosable body 102.

For example, according to one embodiment, the sealing indicator 134 may be configured such that the top edge 135 is obscured only when the cover 104 is sealed to the enclosable body 102. As such, if a user can see the top edge 135 of the sealing indicator 134, then the cover 104 is not sealed to the enclosable body 102, even if the cover 104 is coupled to the enclosable body 102. According to another embodiment, the sealing indicator 134 may be configured such that the bottom edge 137 is obscured only when the cover 104 is sealed to the enclosable body 102. As such, if a user can see the bottom edge 137 of the sealing indicator 134, then the cover 104 is not sealed to the enclosable body 102, even if the cover 104 is coupled to the enclosable body 102.

According to further embodiment, the sealing indicator 134 may be located on a portion of the outer and/or inner surfaces 108, 110 of the enclosable body 102 such that at least one of a top and/or bottom edges 135, 137 is obscured by a portion of the cover 104 only when the cover 104 is sealed to the enclosable body 102, at least one of the top and/or bottom edges 135, 137 is below the mating interface(s) 116a and/or the sealing interfaces 118 of the enclosable body 102.

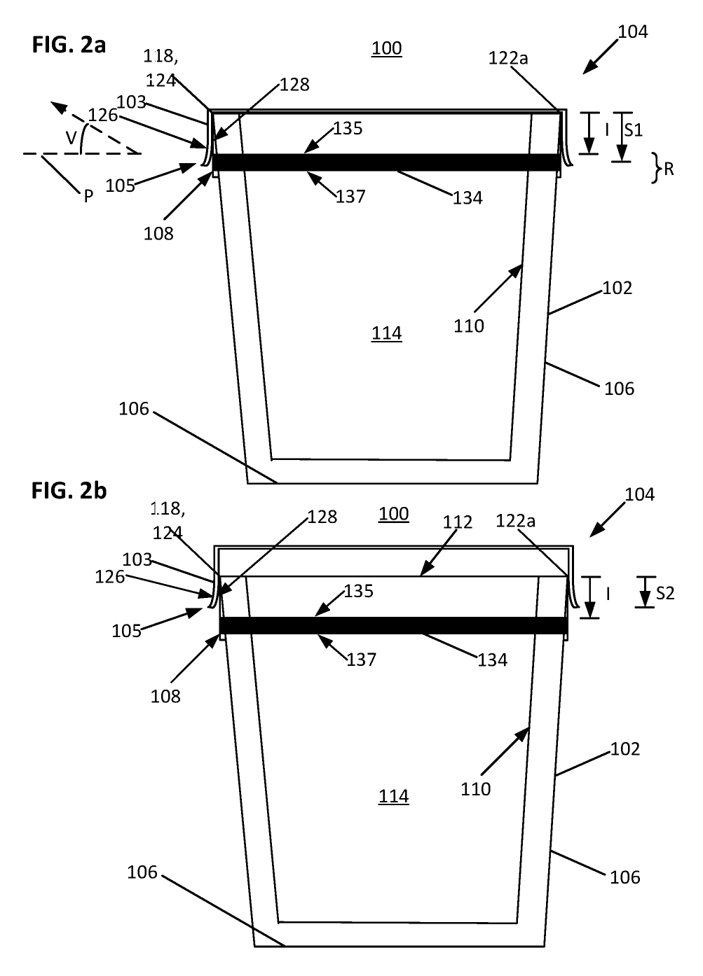

Turning now to FIGS. 2a and 2b, one embodiment of a sealing indicator 134 is generally illustrated. In particular, FIG. 2a generally shows a sealing indicator 134 having a top edge 135 that is obscured by a portion of the cover/lid 104 (e.g., the sidewall 103 and/or flange 105) only when the cover/lid 104 is sealed to the enclosable body 102. For example, the upper edge 135 of the sealing indicator 134 may be disposed a distance I from the opening 112 (e.g., the perimeter and/or edge 122a of the opening 112) along the sidewall 106n. The distance I corresponds to a location which the cover/lid 104 is sealed to the enclosable body 102. When the cover/lid 104 is sealed to the enclosable body 102 (e.g., as shown in FIG. 2a), the sidewall 103 and/or flange 105 of the cover/lid 104 extends a distance Si from the opening 112 (e.g., the perimeter and/or edge 122a of the opening 112) along the sidewall 106n which is equal to, or greater than, distance I. It may be appreciated that the overall length of the sidewall 103 and/or flange 105 may be equal to or greater than the distance S1.

It may be appreciated that the cover/lid 104 may be sealed to the enclosable body 102 in a range R of distances from the opening 112 (e.g., the perimeter and/or edge 122a of the opening 112) along the sidewall 106n. The upper edge 135 of the sealing indicator 134 may therefore be disposed anywhere within the range R, as long as the position of the upper edge 135 corresponds to a position that is obscured by a portion of the cover 104 only when the cover 104 is sealed to the enclosable body 102.

In contrast, FIG. 2b generally shows that the top edge 135 of the sealing indicator 134 is not obscured by the cover/lid 104 when the cover/lid 104 is not sealed to the enclosable body 102. In particular, it can be seen that the sidewall 103 and/or flange 105 of the cover/lid 104 extends a distance S2 from the opening 112 (e.g., the perimeter and/or edge 122a of the opening 112) along the sidewall 106n which is less than distance I. This position therefore generally illustrates the cover/lid 104 in an unsealed state, even though the cover/lid 104 may be coupled to the enclosable body 102. As such, a user and/or machine can quickly and easily identify whether the cover/lid 104 is sealed to the enclosable body 102 based on whether the top edge 135 is visible (e.g., obscured).

While the sealing indicator 134 is shown extending only partially along the sidewall 106 of the enclosable body 102 when the cover/lid 104 is sealed to the enclosable body 102, it should be appreciated that this is not a limitation of the present disclosure unless specifically claimed as such. For example, the sealing indicator 134 may extend all the way to the bottom (e.g., base 106b) of the enclosable body 102. As may be appreciated, the top edge 135 of the sealing indicator 134 is used to determine whether the cover/lid 104 is sealed to the enclosable body 102.

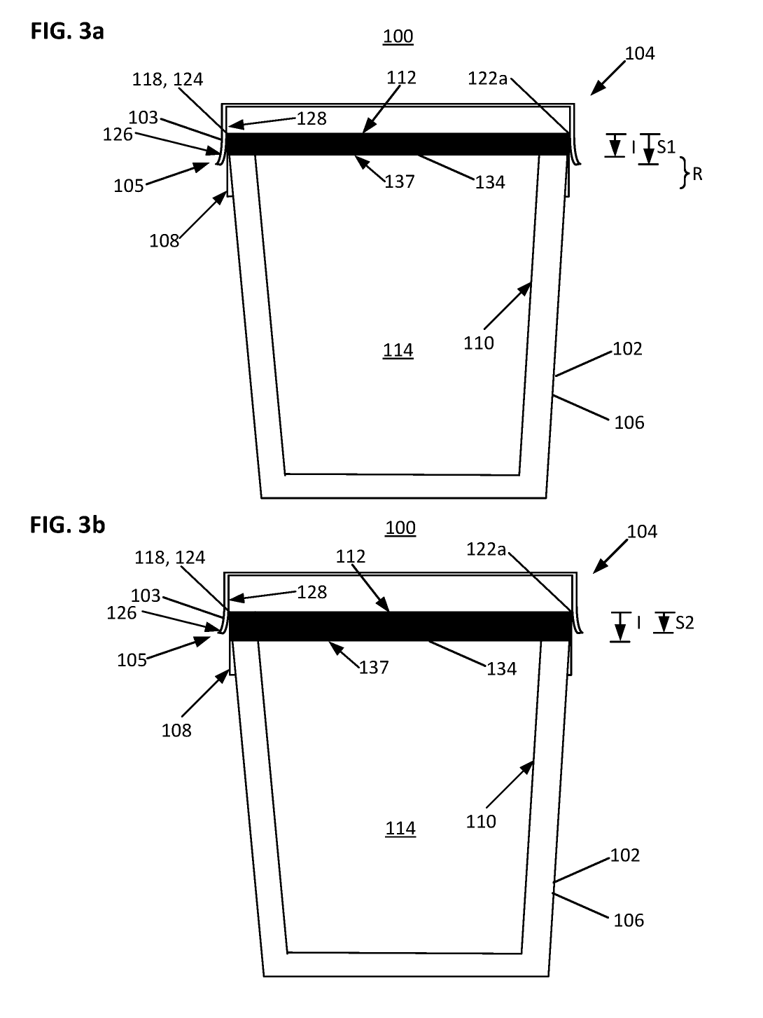

Turning now to FIGS. 3a and 3b, another embodiment of a sealing indicator 134 is generally illustrated. In particular, FIG. 3a generally shows a sealing indicator 134 having a bottom edge 137 that is obscured by a portion of the cover/lid 104 (e.g., the sidewall 103 and/or flange 105) only when the cover/lid 104 is sealed to the enclosable body 102. For example, the bottom edge 137 of the sealing indicator 134 may be disposed a distance I from the opening 112 (e.g., the perimeter and/or edge 122a of the opening 112) along the sidewall 106n. Similar to the embodiment shown and described in FIGS. 2, the distance I corresponds to a location which the cover/lid 104 is sealed to the enclosable body 102. When the cover/lid 104 is sealed to the enclosable body 102 (e.g., as shown in FIG. 3a), the sidewall 103 and/or flange 105 of the cover/lid 104 extends a distance S1 from the opening 112 (e.g., the perimeter and/or edge 122a of the opening 112) along the sidewall 106n which is equal to, or greater than, distance I. It may be appreciated that the overall length of the sidewall 103 and/or flange 105 may be equal to or greater than the distance S1.

It may be appreciated that the cover/lid 104 may be sealed to the enclosable body 102 in a range R of distances from the opening 112 (e.g., the perimeter and/or edge 122a of the opening 112) along the sidewall 106n. The bottom edge 137 of the sealing indicator 134 may therefore be disposed anywhere within the range R, as long as the position of the bottom edge 137 corresponds to a position that is obscured by a portion of the cover 104 only when the cover 104 is sealed to the enclosable body 102.

In contrast, FIG. 3b generally shows that the bottom edge 137 of the sealing indicator 134 is not obscured by the cover/lid 104 when the cover/lid 104 is not sealed to the enclosable body 102. In particular, it can be seen that the sidewall 103 and/or flange 105 of the cover/lid 104 extends a distance S2 from the opening 112 (e.g., the perimeter and/or edge 122a of the opening 112) along the sidewall 106n which is less than distance I. This position therefore generally illustrates the cover/lid 104 in an unsealed state, even though the cover/lid 104 may be coupled to the enclosable body 102. As such, a user and/or machine can quickly and easily identify whether the cover/lid 104 is sealed to the enclosable body 102 based on whether the top edge 135 is visible (e.g., obscured).

While the sealing indicator 134 is shown extending from perimeter and/or edge 122a of the opening 112 along the sidewall 106 of the enclosable body 102 when the cover/lid 104 is sealed to the enclosable body 102, it should be appreciated that this is not a limitation of the present disclosure unless specifically claimed as such. For example, the sealing indicator 134 may extend only along a portion of the sidewall 106n. As may be appreciated, the bottom edge 137 of the sealing indicator 134 is used to determine whether the cover/lid 104 is sealed to the enclosable body 102.

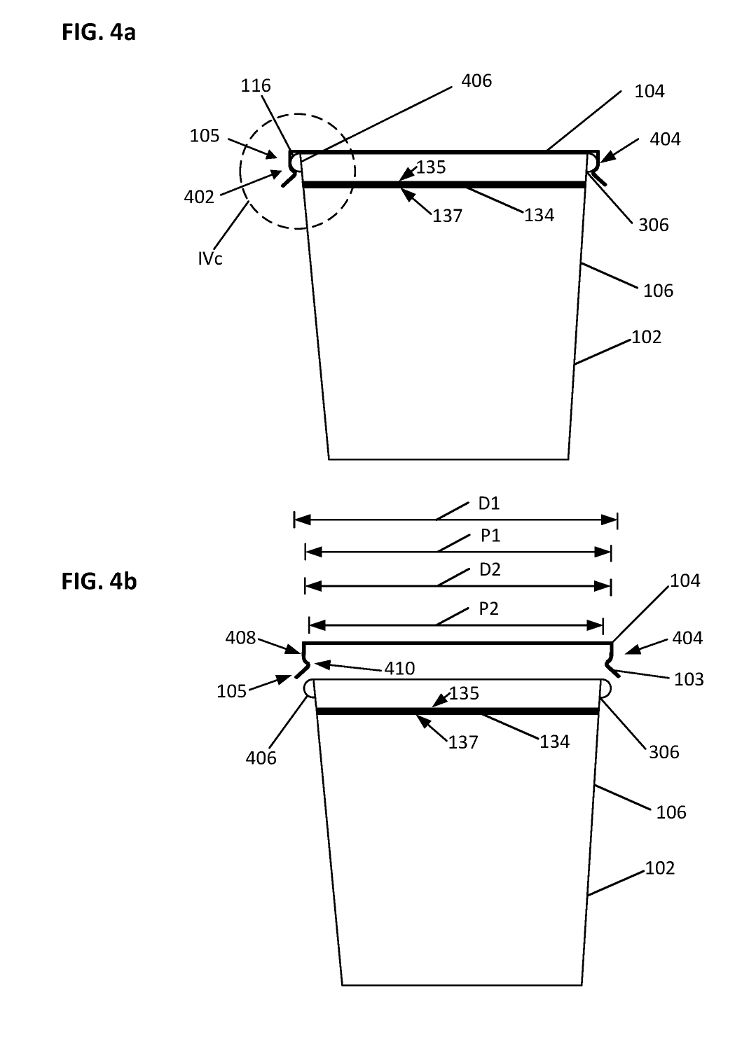

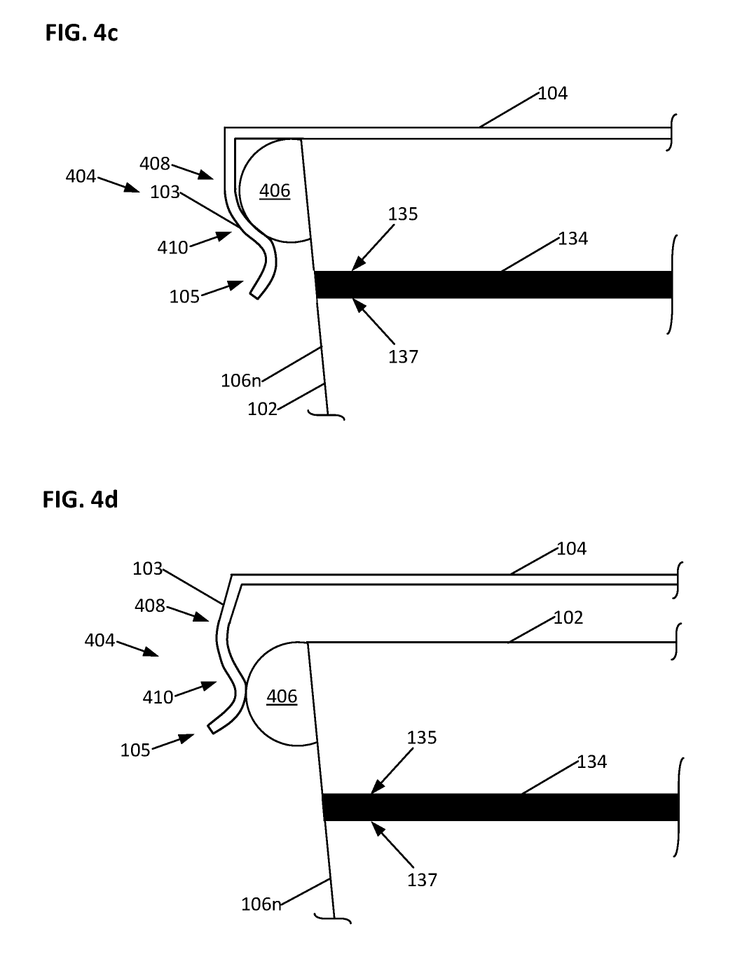

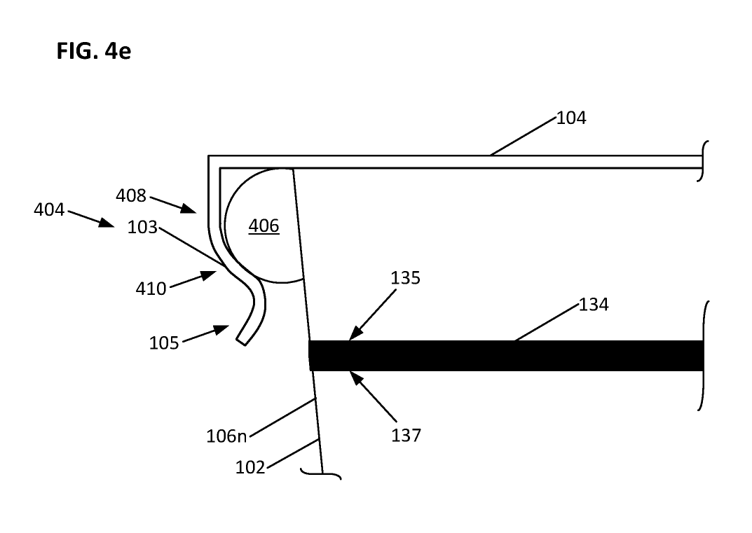

Turning now to FIGS. 4a-4c, yet another embodiment of the sealable container 100 having a sealing indicator 134 is generally illustrated. In particular, FIG. 4a generally illustrates the cover/lid 104 is sealed to enclosable body 102 with a snap-fit 402 while FIG. 4b generally illustrates the cover/lid 104 and enclosable body 102 in an unassembled state. FIG. 4c generally illustrates an expanded region IVc in FIG. 4a showing the cover/lid 104 is sealed to enclosable body 102 with a snap-fit 402 while FIG. 4d generally illustrates an expanded region generally corresponding to the region IVc in FIG. 4a showing the cover/lid 104 and enclosable body 102 in an unsealed state. It should be appreciated that the cover/lid 104 and enclosable body 102 in FIG. 4d may be coupled and/or partially coupled to the enclosable body 102 (but not sealed).

As may be appreciated, the snap fit 402 may be formed when an engagement portion 404 of the cover/lid 104 is resiliently deformed and/or expanded over and/or around at least a portion 406 of the enclosable body 102. For example, the portion 406 of the enclosable body 102 may include a circumferential and/or peripheral lip or protrusion extending generally radially outward proximate to the opening 112 of the cavity 114. In the illustrated embodiment, the lip or protrusion 406 may have a generally hemispherical cross-sectional shape. The lip or protrusion 406 may also form the outer and/or inner edges 122a, 122b of the sidewall 106 of the enclosable body 102.

With reference to FIG. 4b, the engagement portion 404 of the cover/lid 104 may include a portion of the sidewall 103. For example, the sidewall 103 may include a first portion 408 and at least a second portion 410 extending generally downwardly from the first portion 408. The first portion 408 may extend generally from the upper, top, and/or central region 107 of the cover/lid 104. At least a portion of the first portion 408 may include an interior dimension P1 (e.g., circumference) that is approximately equal to (or slightly larger than) the largest external dimension D2 (e.g., circumference) of the lip or protrusion 406. At least a portion of the second portion 410 may include an interior dimension P2 (e.g., circumference) that is smaller than both dimensions P1 and D2. The sidewall 103 may also optionally include a flange 105 having an interior dimension D1 (e.g., circumference) that larger than dimension D2 (e.g., circumference) of the lip or protrusion 406.

Turning now to FIG. 4c, an expanded view of region IVc of FIG. 4a is generally illustrated showing the cover/lid 104 is sealed to enclosable body 102 with a snap-fit 402. As can be seen, the first and/or second portions 408, 410 of the sidewall 103 have resiliently deformed around the lip or protrusion 406 of the enclosable body 102. At least a portion of the second portion 410 engages and/or contacts against a portion of the lip or protrusion 406 of the enclosable body 102 to seal the cover/lid 104 to the enclosable body 102. In particular, the smaller dimension P2 of the second portion 410 extends around and/or over a portion of the lip or protrusion 406 to create a generally downward force that urges the cover/lid 104 against a portion of the sidewall 106n of the enclosable body 102.

As can be seen, the sidewall 103 partially obscures the lower edge 137 of the sealing indicator 134 when the cover/lid 104 is sealed to the enclosable body 102. While the lower edge 137 of the indicator 134 is shown being obscured when the cover/lid 104 is sealed to the enclosable body 102, it should be appreciated that the upper edge 135 of the indicator 134 may be obscured when the cover/lid 104 is sealed to the enclosable body 102.

Turning now to FIG. 4d, an expanded region generally corresponding to the region IVc in FIG. 4a is generally illustrated showing the cover/lid 104 and enclosable body 102 in an unsealed state. As can be seen, the sidewall 103 is deformed about the lip or protrusion 406 of the enclosable body 102. While the sidewall 103 may obscure the lip or protrusion 406, the cover/lid 104 is not sealed to the enclosable body 102, even though the cover/lid 104 may be coupled to the enclosable body 102. As such, the lip or protrusion 406 does not function as a sealing indicator 134 as defined herein since the lip or protrusion 406 is not obscured only when a seal is formed.

Turning now to FIG. 4e, another embodiment showing an expanded region similar to region IVc in FIG. 4a is generally illustrated showing the cover/lid 104 and enclosable body 102 in a sealed state. While the cover 104 in FIGS. 4a-4d is generally illustrated obscuring the bottom edge 137 of the sealing indicator 134, the cover 104 in FIG. 4e is generally illustrated obscuring the top edge 135 of the sealing indicator 134. As such, a user can determine that the cover 104 is sealed to the enclosable body 102 if the top edge 135 is obscured. For example, if a user can see a portion of the enclosable body 102 above the top edge 135 of the sealing indicator 134, then the cover 104 is not sealed to the enclosable body 102.

In the illustrated embodiment, the cover/lid 104 may, at least partially, be made of a resiliently deformable material (e.g., plastics, silicones, elastomers, and etc . . . ). In other embodiments, the enclosable body 102 may be made, at least partially, of a resiliently deformable material. In such an embodiment, the cover/lid 104 may be substantially rigid and the enclosable body 102 may deform in response to the cover/lid 104 being forcibly positioned on the enclosable body 102.

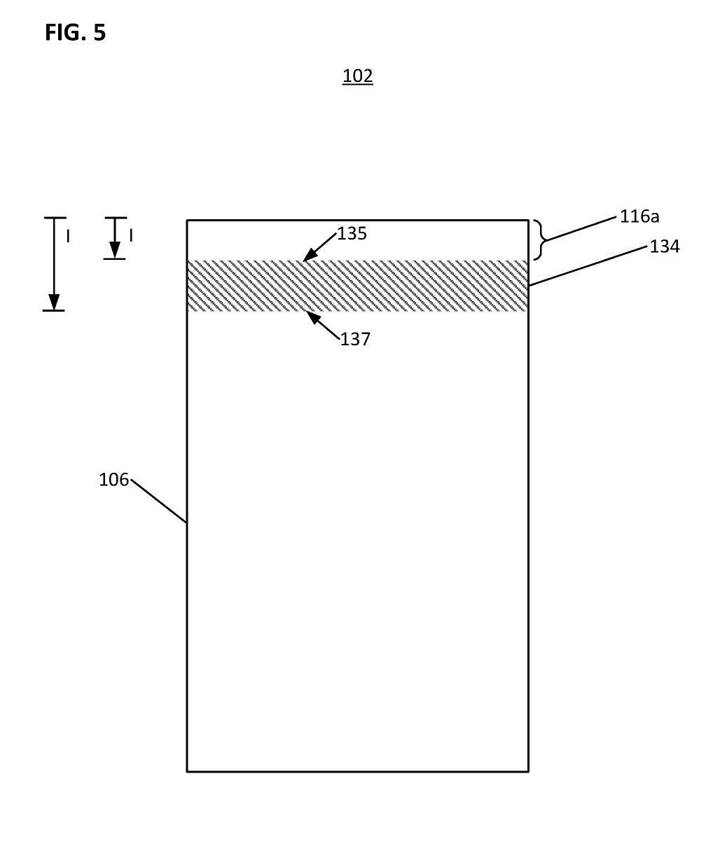

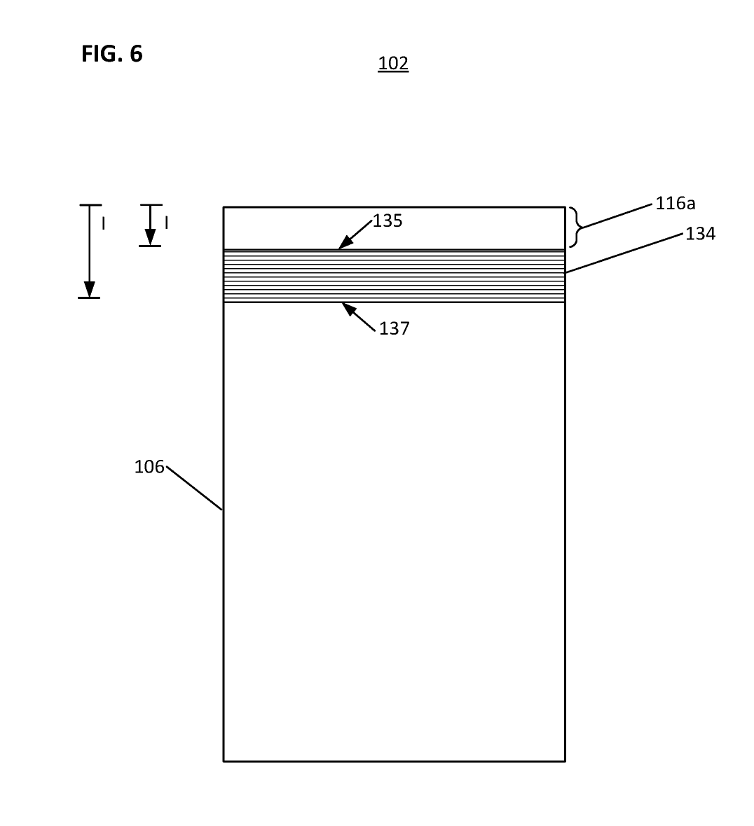

Referring also to FIGS. 5-9, various embodiments of the sealing indicator 134 are generally illustrated. For example, the sealing indicator 134 may include a hashed design (see, e.g., FIG. 5). As may be seen, the upper and lower edges 135, 137 of the hashed sealing indicator 134 may be formed by the ends of the hashes. Alternatively (or in addition), the sealing indicator 134 may include a gradient (see, e.g., FIG. 6). The gradient sealing indicator 134 may include a plurality of indicia (e.g., gradients) representing a degree of seal (e.g., the strength of the seal and/or connection formed between the cover/lid 104 and the enclosable body 102) using at least two lines or indicia, a change in shading, a change in color, or some other like measure of change/variation.

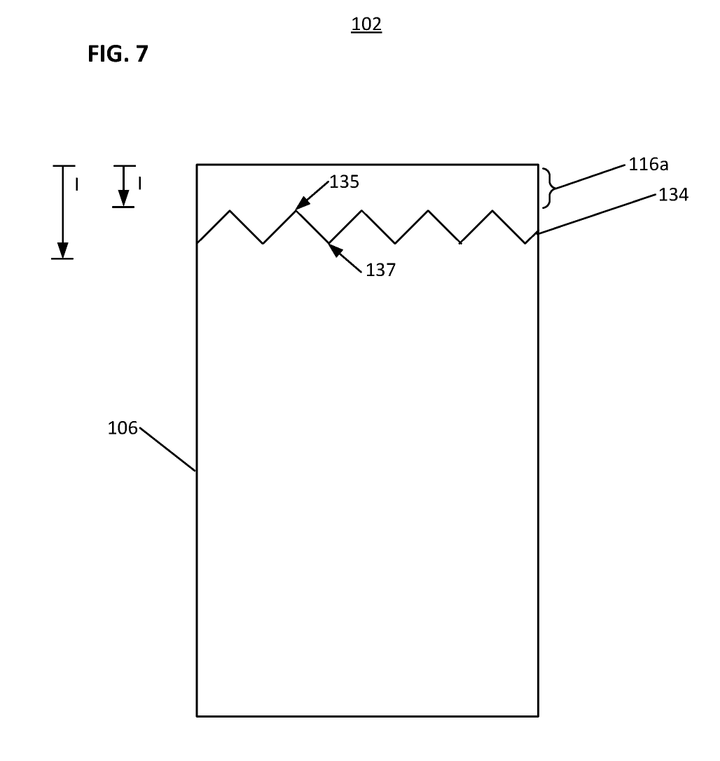

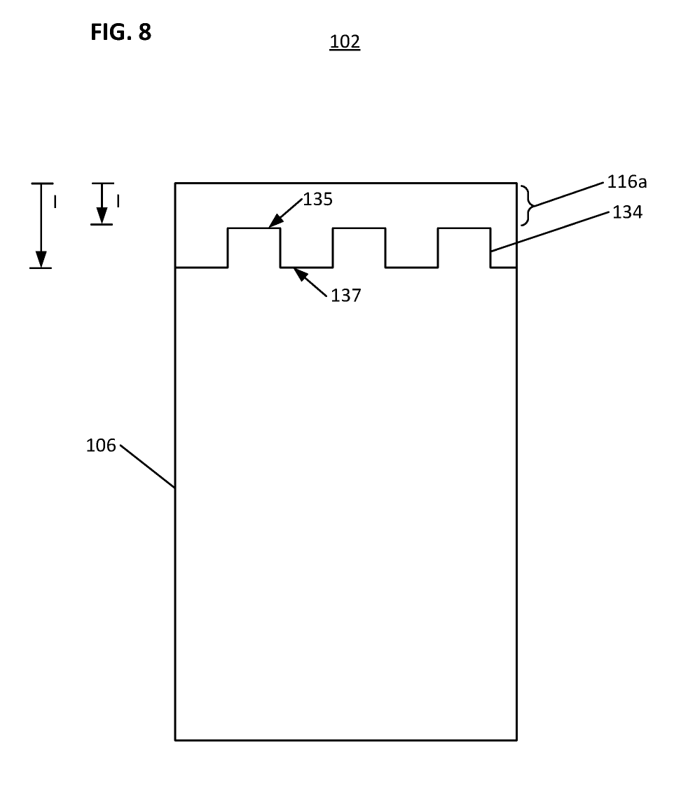

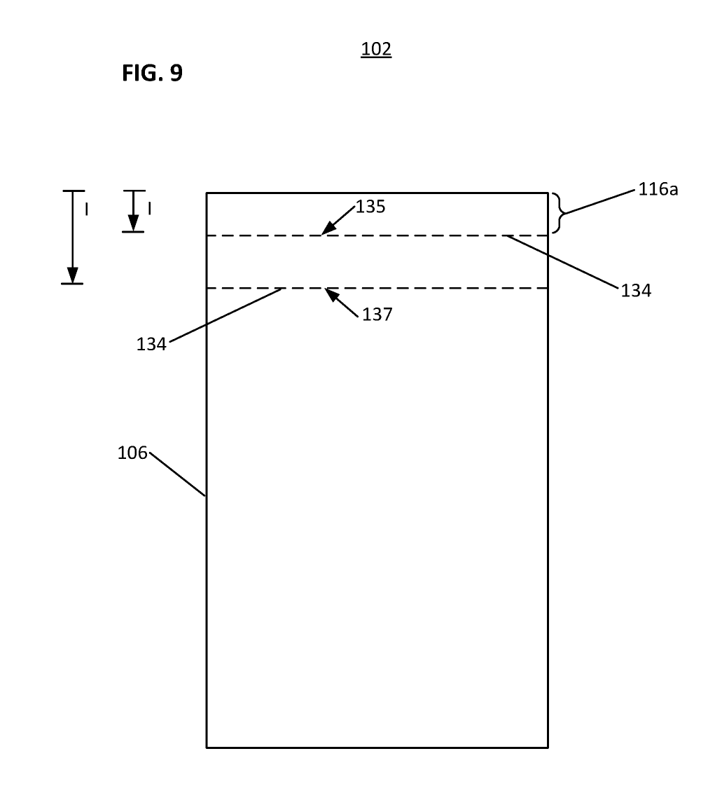

The sealing indicator 134 may include a jagged line (see e.g., FIG. 7) or a square (or sinusoidal) wave (see, e.g., FIG. 8) in which portions of the indicator form the upper and lower edges 135, 137. The sealing indicator 134 may include one or more non-continuous lines (see, e.g., FIG. 9), or any other shape or form. Further, the sealing indicator 134 may be any form of marking including, but not limited to, a physical indentation(s), a printed line(s), a painted line(s), a ribbon(s), a gasket(s), a groove(s), a rib(s), a protrusion(s), any combination of printed lines, painted lines, ribbons, gaskets, grooves, ribs, protrusions, and any other form of marking an object.

Any of the sealing indicators 134 described herein may extend around all, or part, of the sidewall 106 (e.g., the outer surface 108). For example, the sealing indicator 134 may extend continuously and/or discontinuously around the sidewall 106n.

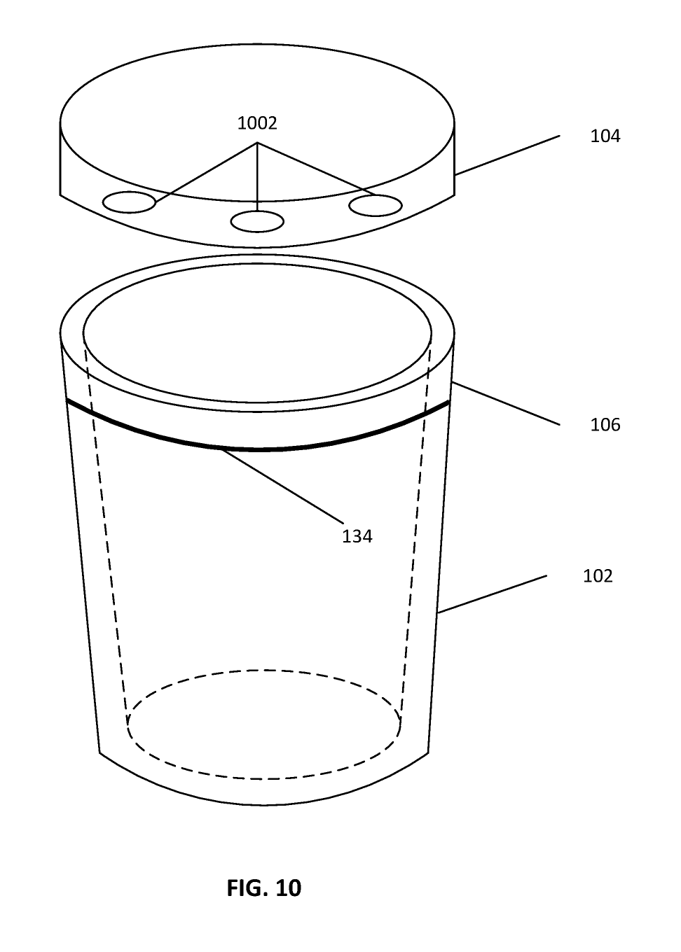

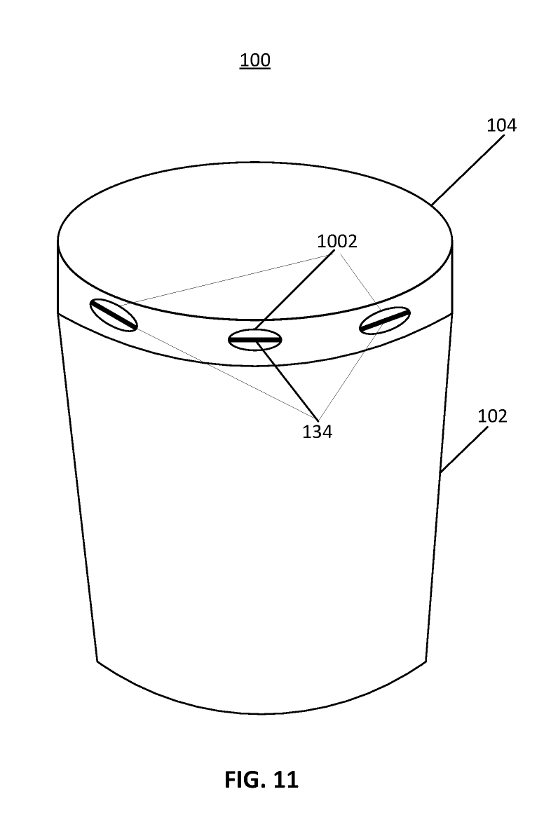

In another embodiment, as shown in FIGS. 10-13, the cover/lid 104 may include at least one inspection element 1002 to reveal all, or part, of the sealing indicator 134 when the enclosable body 102 is sealed to the cover/lid 104. For example, with reference to FIG. 10, the sealing indicator 134 may be disposed on the enclosable body 102 (e.g., on a portion of the outer and/or inner surfaces 108, 110 of the enclosable body 102) such that at least one of a portion (e.g., but not limited to, a top and/or bottom edges 135, 137) of the sealing indicator 134 is viewable through the inspection element 1002 only when the cover 104 is sealed to the enclosable body 102, for example, as generally illustrated in FIG. 11. As such, a user and/or machine may quickly and easily verify that the cover 104 is sealed to the enclosable body 102 if a portion of the sealing indicator 134 is visible through the inspection element 1002. It may be appreciated that the sealing indicator 134 may or may not be visible to the human eye, for example, it may be only visible to a machine sensor or the like.

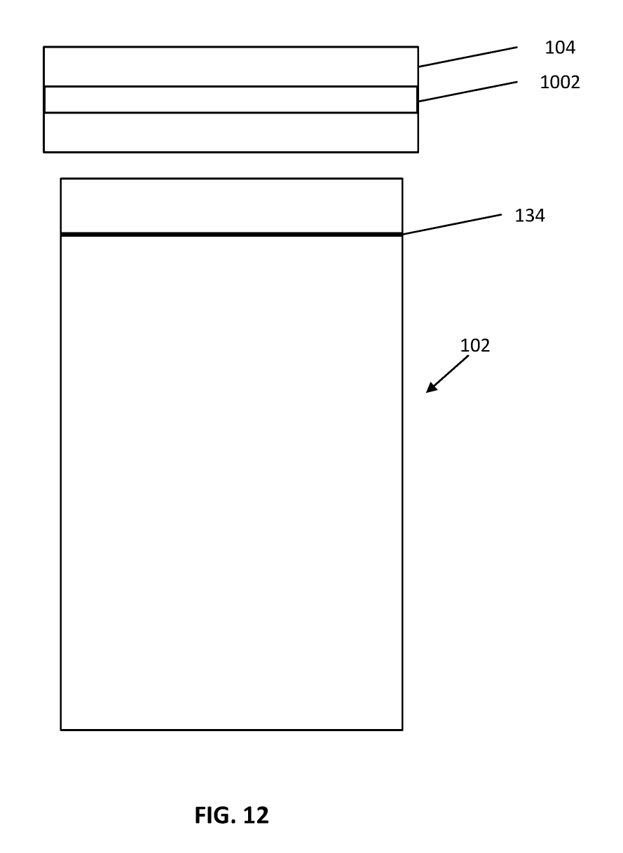

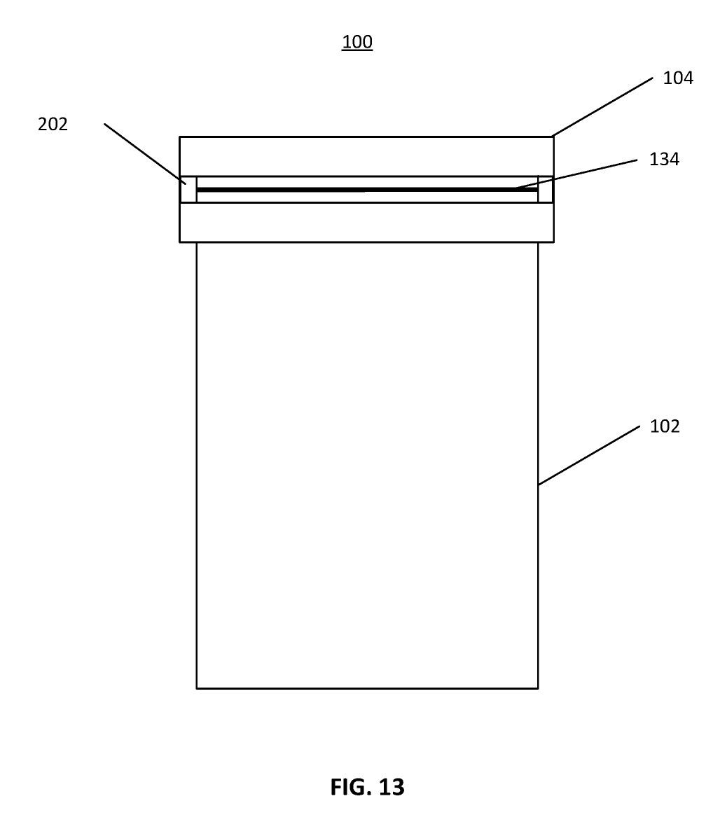

The inspection element(s) 1002 may take any shape reasonably capable revealing a portion of the sealing indicator 134 when the cover/lid 104 is sealed to enclosable body 102. The inspection element 1002 may be a hole cut-out, passageway, and/or a through-hole in cover/lid 104 (see, e.g., FIG. 11). According to one embodiment, the inspection element 1002 may include a clear material or a transparent material disposed within at least a portion of the hole cut-out, passageway, and/or through-hole. In another example, as shown in FIG. 12, the inspection element 1002 may be a clear band contained within the cover/lid 104. In this example, the cover/lid 104 seals the enclosable body 102 when at least a portion of the sealing indicator 134 can be observed in and/or fills the clear band (see, e.g., FIG. 13). In another example, the cover/lid 104 may be made of a transparent, or semi-transparent, material such that the sealing indicator 134 can be at least partially observed through the cover/lid 104. In this situation, the cover/lid 104 may have a marking disposed on it that is positioned below, above, or in-line with the sealing indicator 134 when the cover/lid 104 is sealed to the enclosable body 102. Whenever a transparent or clear material is used, the inspection element 1002 (and/or the cover/lid 104) may be colored such that when the inspection element 1002 (and/or the cover/lid 104) reveals at least a portion of the enclosed and/or the seal indicator 134, it at least partially changes color (e.g., if the sealing indicator 134 is red and the inspection element 1002 contains a transparent green material, the inspection element 1002 would display a yellow color when the enclosable body 102 is sealed and/or enclosed)

By way of further example, the cover/lid 104 and the enclosable body 102 may both be transparent. In this example, the cover/lid 104 may include a first polarized section and the enclosable body 102 may include a second polarized section, wherein each polarized section is configured such that substantially no light is transmitted through the polarized sections when the cover/lid 104 is sealed to the enclosable body 102. Alternatively (or additionally), the cover/lid 104 and the enclosable body 102 may each include a different colored transparent or semi-transparent material such that there is a change in color when the enclosable body 102 is sealed to the cover/lid 104 (e.g., if the cover/lid 104 is red and the enclosable body 102 is green, a yellow color would appear when the enclosable body 102 is sealed and/or enclosed).

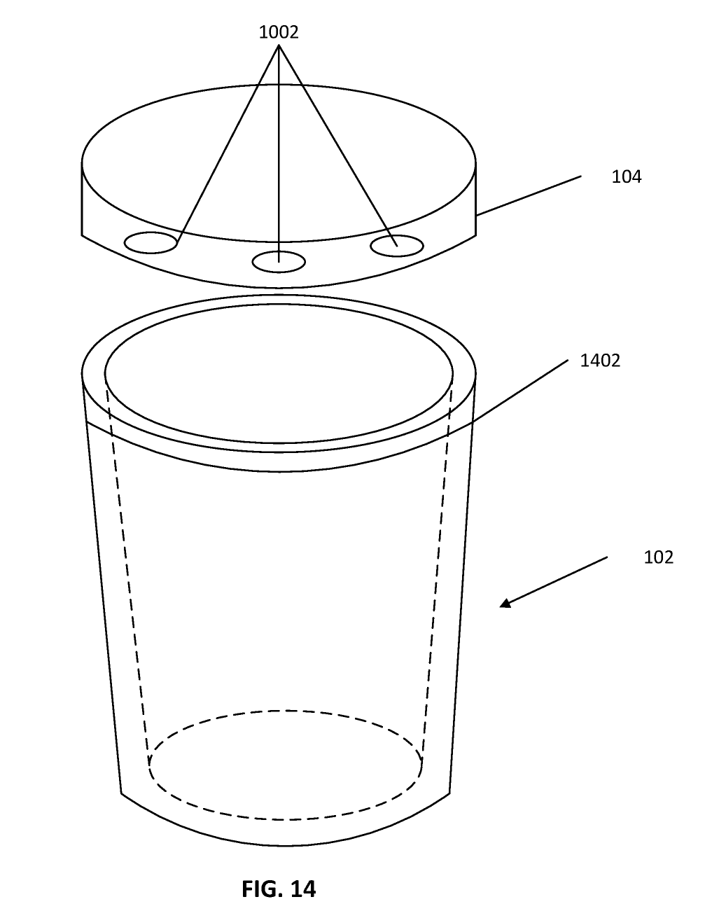

As may be appreciated, the cover/lid 104 may be positioned on the enclosable body 102 without forming a seal (e.g., the cover/lid 104 may be coupled to the enclosable body 102 without the cover/lid 104 being sealed to the enclosable body 102). As such, there would be partial engagement between the enclosable body 102 and the cover/lid 104. As shown in FIG. 14, the use of the inspection element 1002 allows for an unsealed indicator 1402 to be provided. The unsealed indicator 1402 indicates the presence of a partial engagement to the user (or a machine), i.e., that the cover/lid 104 is not sealed to the enclosable body 102. For example, when the unsealed indicator 1402 is visible, a seal is not formed between the enclosable body 102 and the cover/lid 104 (for example, because there is a partial engagement between the enclosable body 102 and the cover/lid 104), even though the cover/lid 104 may (or may not) be coupled to the enclosable body 102. To provide adequate notification of partial engagement, the unsealed indicator 1402 is positioned such that the unsealed indicator 1402 is visible through the inspection elements 1002 when the cover/lid 104 is engages the enclosable body 102, but not when the cover/lid 104 is sealed to the enclosable body 102.

It should be appreciated that any sealable container 100 described herein may include both a sealing indicator 134 and an unsealed indicator 1402. The space between the unsealed indicator 1402 and the sealing indicator 134 may be representative of a degree of seal and/or enclosure.

In order to facilitate inspection through inspection element 1002, unsealed indicator 1402 may be any form of marking including, but not limited to, those markings described in relation to the sealing indicator 134, a physical indentation(s), a printed line(s), a painted line(s), a ribbon(s), a gasket(s), a groove(s), a rib(s), a protrusion(s), any combination of those markings described in relation to the sealing indicator 134, printed lines, painted lines, ribbons, gaskets, grooves, ribs, protrusions, and any other form of marking an object. As should be appreciated, the inspection element 1002 may be used to reveal the unsealed indicator 1402 in substantially the same manner as was discussed previously in relation to the sealing indicator 134.

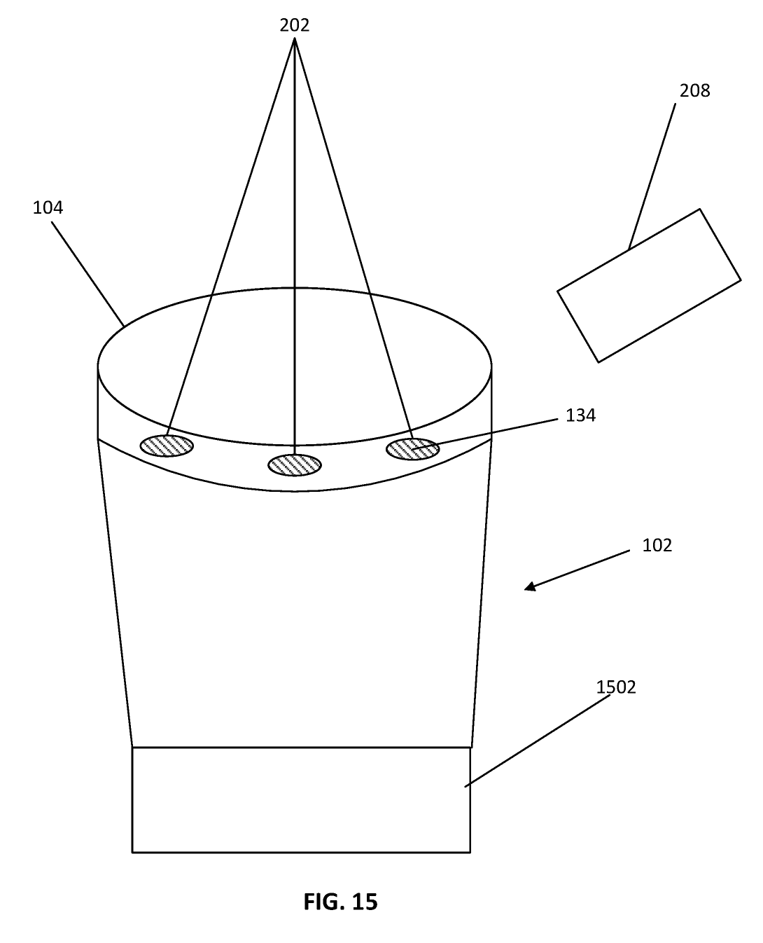

As shown in FIG. 15, and according to another aspect, the inspection element 1002, may be used for machine verification of the formation of a seal between the enclosable body 102 and the cover/lid 104. For example, the inspection element 1002 may be used in conjunction with a sensor 208, e.g., an optical sensor or the like. In this situation, for example, the optical sensor 208 would be configured to detect the presence of one or more sealing indicators 134 within the inspection element 1002. When the sealing indicator 134 is detected, the optical sensor 208, in combination with a computer or microcontroller, may perform an action (e.g., a visual prompt, an audible alarm, a movement of enclosable body 102 to a secured covering element location, and etc . . . ) indicating that the cover/lid 104 is properly sealing and/or enclosing the enclosable body 102. Alternatively, when the sealing indicator 134 is not detected, the optical sensor 208, in combination with a computer or microcontroller, may perform an action (e.g., a visual prompt, an audible alarm, a movement of enclosable body 102 to an unsecured covering element location, and etc . . . ) indicating that the cover/lid 104 is not properly sealing or enclosing the enclosable body 102. In some embodiments, a retaining device 1502 may be used to position the enclosable body 102 such that the optical sensor 208 may sense the inspection element 1002. As may be appreciated, the above described machine verification may be substantially applied to embodiments employing the unsealed indicator 1402 (not shown).

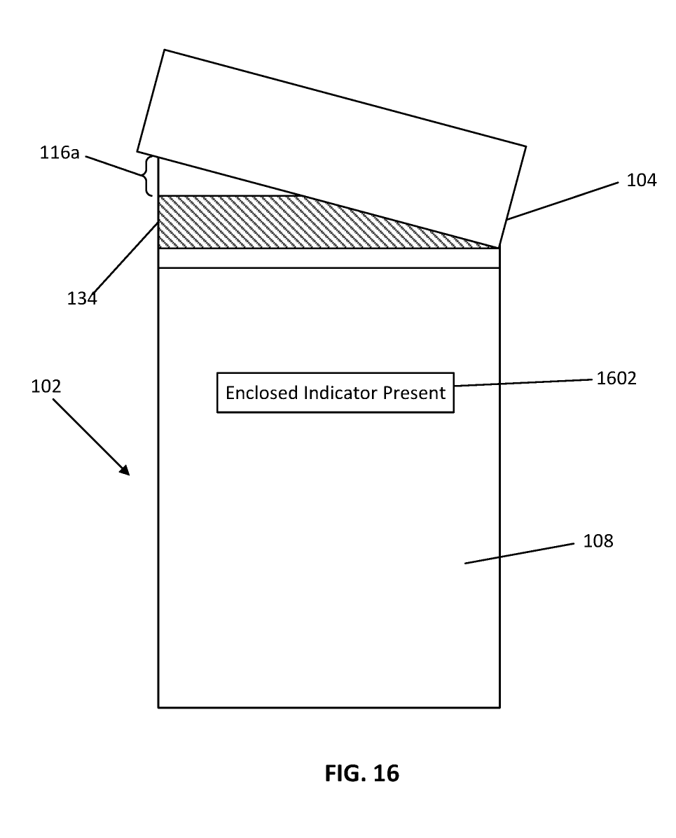

Referring also to FIG. 16, the cover/lid 104 may partially engage a first portion of mating interface 116a and fully engage a second portion of mating interface 116a. Partial engagement may be indicated by observing a portion of the sealing indicator 134. In this situation, a 360-degree visual inspection of the enclosable body 102 may be necessary. A 360-degree visual inspection may involve rotating the enclosable body 102. Alternatively (or additionally), a mirror may be positioned around the enclosable body 102 such that all sides of the enclosable body 102 may be observable from one position.

In some embodiments, the final inspection may be performed by an end user of enclosable body 102. As may be appreciated, the end user may not be aware of the presence of the sealing indicator 134. In this situation, the end user may either assume the sealing indicator 134 is present and covered by the cover/lid 104 or the end user may remove the cover/lid 104 and verify if the sealing indicator 134 is present. To address this situation, some embodiments may include a certification indicator 1602 to alert the end user to the presence of the sealing indicator 134. In some embodiments, the certification indicator 1602 may be located on the outer surface 108 of the enclosable body 102 (as shown) and/or on the outer surface of the cover/lid 104. The certification indicator 1602 may be any marking (e.g., ribs, protrusions, paint, ink, and etc . . . ) sufficient to alert the end user of the presence of the sealing indicator 134, despite sealing indicator 134 potentially being completely covered.

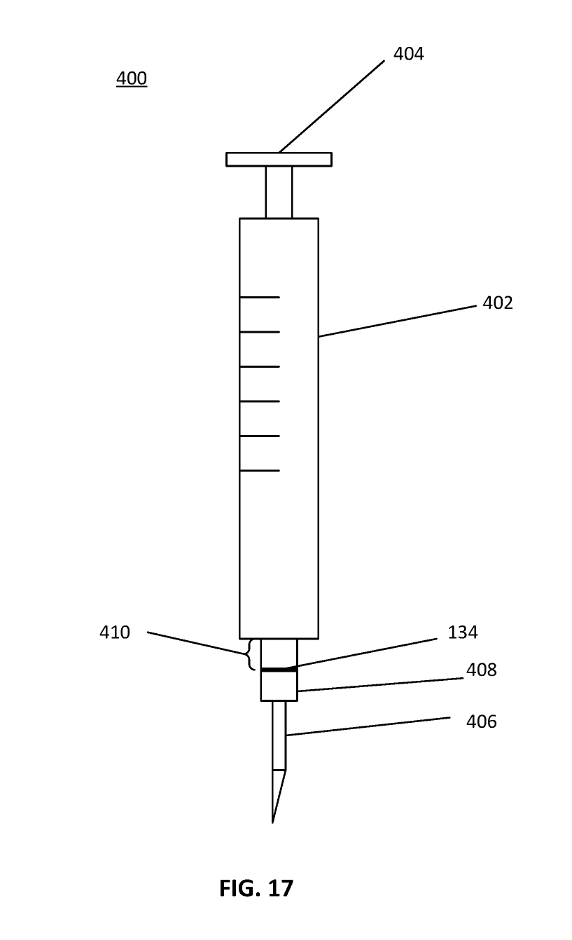

Referring also to FIG. 17, the principals of the prior discussion may be substantially applied to a syringe 400 that includes a syringe body 402, a depressing mechanism 404, a needle 406, and a needle cap 408. The needle cap 408 may be internally and/or externally threaded (e.g., as in a Luer lock syringe). Alternatively, the needle cap 408 may not be threaded and may rely on a friction and/or interference fit to form a seal (e.g., as in a slip tip syringe). The syringe body 402 may be transparent or semi-transparent. The needle cap 408 may threadably connect to the engagement region 410 of the syringe body 402. The engagement region 410 may include a sealing indicator 134. The sealing indicator 134 may be any form of marking including, but not limited to, a physical indentation(s), a printed line(s), a painted line(s), a ribbon(s), a gasket(s), a groove(s), a rib(s), a protrusion(s), any combination of printed lines, painted lines, ribbons, gaskets, grooves, ribs, protrusions, and any other form of marking an object as described herein.

If the needle cap 408 is internally threaded (or forms a friction and/or interference fit with an external surface of the engagement region 410), a predefined portion of the sealing indicator 134 may be covered by the needle cap 408 when the needle cap 408 is sealed to the engagement region 410. Alternatively, if the needle cap 408 is externally threaded (or forms a friction and/or interference fit with an internal surface of engagement region 410), a second sealing indicator may be disposed on the needle cap 408 such that when sealed, the sealing indicator 134 is aligned with, is positioned above, and/or is positioned below the second sealing indicator. For example, when the second sealing indicator aligns with the sealing indicator 134 (i.e., only one indicator is visible), the needle cap 408 seals the syringe body 402.

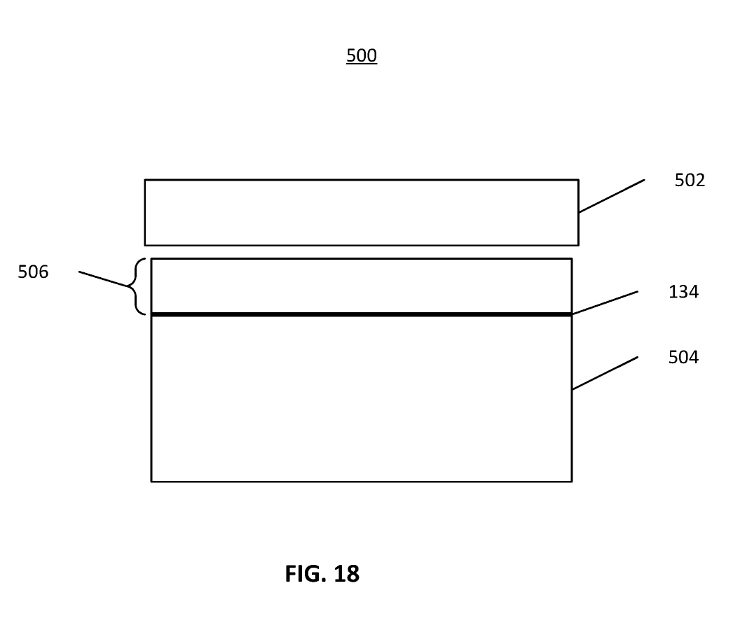

Referring also to FIG. 18, the principals of the prior discussion may be substantially applied to a container or box 500, which may include a top 502 and a body 504. The box 500 may take any shape including rectangular, octagonal, square, pentagonal, triangular, circular, and other geometric shapes. The top 502 may slide over the mating interface 506, forming a friction and/or interference fit between the mating interface 506 and the top 502. The box 500 may further include a sealing indicator 134. The sealing indicator 134 may be any form of marking including, but not limited to, a physical indentation(s), a printed line(s), a painted line(s), a ribbon(s), a gasket(s), a groove(s), a rib(s), a protrusion(s), any combination of printed lines, painted lines, ribbons, gaskets, grooves, ribs, protrusions, and any other form of marking an object as described herein.

The sealing indicator 134 may indicate the desired position of the top 502 such that the top 502 engages the mating interface 506 in a manner sufficient to retain the contents of the box 500 (e.g., generally sealed). The contents of the box 500 may be a solid, a liquid, and/or a gas. In order to determine if the top 502 is in the desired position, the top 502 may cover a predefined portion of the sealing indicator 134. In some instances, the predefined portion may be the entirety of the sealing indicator 134.

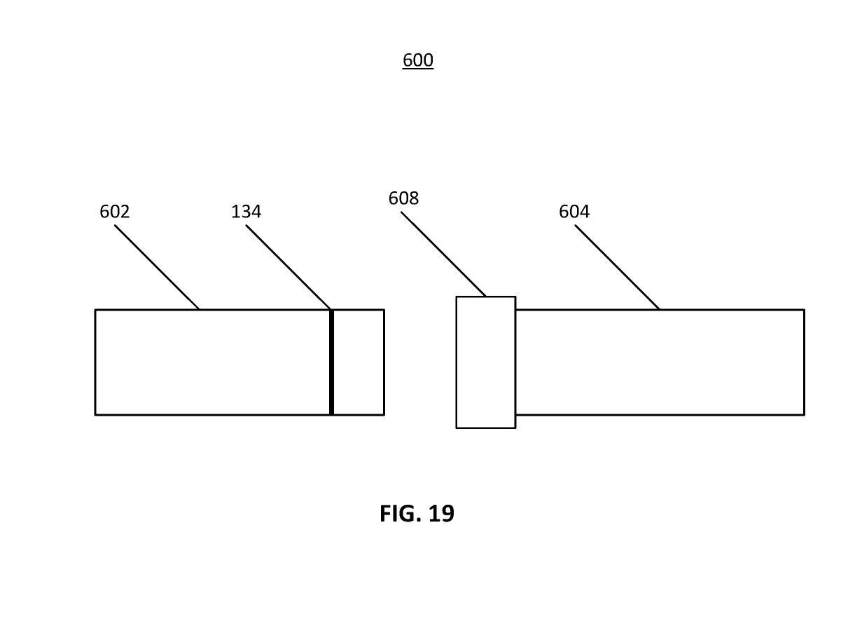

Referring also to FIG. 19, the principals of the prior discussion may be substantially applied to a pipe system 600. The pipe system 600 may include a male pipe 602 and a female pipe 604. The male pipe 602 may include a sealing indicator 134. The sealing indicator 134 may be any form of marking including, but not limited to, a physical indentation(s), a printed line(s), a painted line(s), a ribbon(s), a gasket(s), a groove(s), a rib(s), a protrusion(s), any combination of printed lines, painted lines, ribbons, gaskets, grooves, ribs, protrusions, and any other form of marking an object as described herein. Regardless of the form of marking, the sealing indicator 134 may represent, for example, the degree of insertion necessary to form a seal between the pipe sections 602, 604 (for example, with the application of an adhesive or sealant).

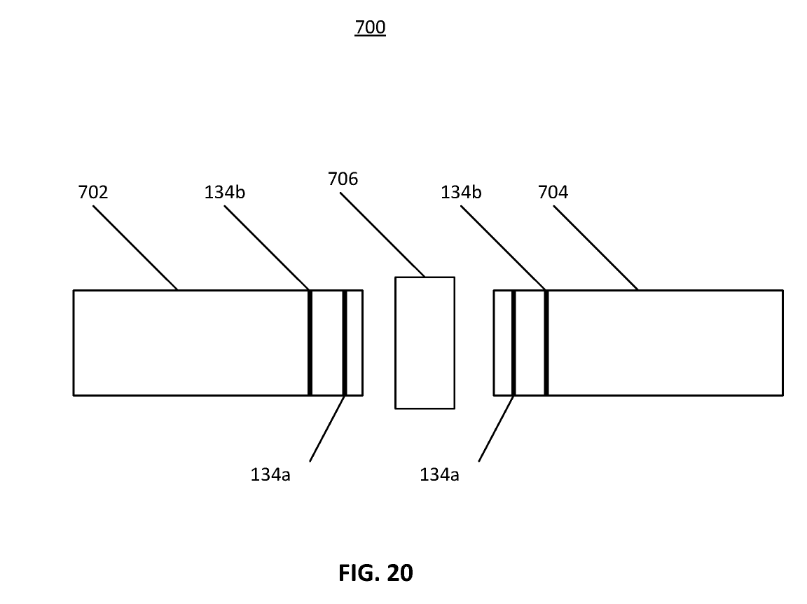

The female pipe 604 may include a coupler 608 for joining the female pipe 604 to the male pipe 602. However, as one having ordinary skill in the art would appreciate, the male pipe 602 may be joined to another male pipe using a coupling (see, e.g., FIG. 20). When mating the female pipe 604 to the male pipe 602, a specific degree of fit may be required. The specific degree of fit may be indicated by the sealing indicator 134. For instance, a secure fit may be indicated if a predefined quantity of the sealing indicator 134 is covered by the coupler 608. The predefined quantity may be the entirety of the sealing indicator 134. Alternatively, the sealing indicator 134 may be a gradient (i.e., two or more markings) wherein each gradient line represents the degree of insertion required. For example, each gradient line may represent a degree of insertion necessary for a specific type of adhesive to securely couple male pipe 602 to female pipe 604. Alternatively (or additionally), if the sealing indicator 134 is a gradient, the first gradient line may represent a minimum insertion distance and the last gradient line may represent a maximum insertion distance. If the maximum insertion distance is exceeded the pipe system 600 may not function properly.

By way of further example, and referring also to FIG. 20, a pipe system 700 may include a first male pipe 702, a second male pipe 704, and a coupler 706. The first male pipe 702 may be coupled to the second male pipe 704 using the coupler 706. The first male pipe 702 may include a minimum sealing indicator 134a and a maximum sealing indicator 134b. The second male pipe 704 may also include the minimum sealing indicator 134a and the maximum sealing indicator 134b. When the first male pipe 702 is properly coupled to the second male pipe 704 using the coupler 706, the coupler 706 will cover the minimum sealing indicator 134a but not the maximum sealing indicator 134b for both the first male pipe 702 and the second male pipe 704.

According to one aspect, the present disclosure features an enclosable body comprising at least one sidewall defining a cavity having an opening, a first mating interface disposed on the at least one body-sidewall substantially adjacent to the opening, a first sealing interface disposed on the at least one body-sidewall, and a sealing indicator disposed within on the at least one sidewall, the sealing indicator configured to indicate that a cover is sealed to the enclosable body.

Optionally, the sealing indicator comprises at least one upper or lower edge, wherein at least one of the upper or lower edges is obscured only when the cover is sealed to the enclosable body.

Optionally, the upper edge of the sealing indicator is obscured only when the cover is sealed to the enclosable body.

Optionally, the lower edge of the sealing indicator is obscured only when the cover is sealed to the enclosable body.

Optionally, at least one of the upper or lower edges is obscured by the cover only when the cover is sealed to the enclosable body.

Optionally, at least one of the upper or lower edges contacts the cover only when the cover is sealed to the enclosable body.

Optionally, a portion of the cover extends over and obscures at least one of the upper or lower edges contacts the cover only when the cover is sealed to the enclosable body.

According to another aspect, the present disclosure features a container system comprising a cover and an enclosable body. The cover includes a central region, at least one sidewall extending from the central region, a first mating interface, and a first sealing interface. The enclosable body comprises at least one sidewall defining a cavity having an opening, a second mating interface disposed on the at least one sidewall, the second mating interface engaging the first mating interface, a second sealing interface disposed on the at least one sidewall, the second mating interface engaging the first sealing interface to form a seal therebetween, and a sealing indicator disposed within on the at least one sidewall, the sealing indicator configured to indicate that the cover is sealed to the enclosable body.

According to yet another aspect, the present disclosure features a covering element comprising a central region, at least one sidewall extending from said central region, a mating and a sealing interface configured to engage with a mating and sealing interface of an enclosable body to form a seal therebetween, and at least one inspection element disposed in said at least one sidewall, said inspection element configured to reveal a sealing indicator of said enclosable body representing that said cover is sealed to said enclosable body.

While the principles of the invention have been described herein, it is to be understood by those skilled in the art that this description is made only by way of example and not as a limitation as to the scope of the invention. Other embodiments are contemplated within the scope of the present invention in addition to the exemplary embodiments shown and described herein. Modifications and substitutions by one of ordinary skill in the art are considered to be within the scope of the present invention, which is not to be limited except by the following claims.

What is claimed is:

1. An enclosable body comprising:
at least one sidewall defining a cavity having an opening;
a first mating interface disposed on said at least one body-sidewall substantially adjacent to said opening;
a first sealing interface disposed on said at least one body-sidewall; and
a sealing indicator disposed within on said at least one sidewall, said sealing indicator configured to indicate that a cover is sealed to said enclosable body.

2. The enclosable body of claim 1, wherein said sealing indicator comprises at least one upper or lower edge, wherein at least one of said upper or lower edges is obscured only when said cover is sealed to said enclosable body.

3. The enclosable body of claim 2, wherein said upper edge of said sealing indicator is obscured only when said cover is sealed to said enclosable body.

4. The enclosable body of claim 2, wherein said lower edge of said sealing indicator is obscured only when said cover is sealed to said enclosable body.

5. The enclosable body of claim 2, wherein at least one of said upper or lower edges is obscured by said cover only when said cover is sealed to said enclosable body.

6. The enclosable body of claim 2, wherein at least one of said upper or lower edges contacts said cover only when said cover is sealed to said enclosable body.

7. The enclosable body of claim 2, wherein a portion of said cover extends over and obscures at least one of said upper or lower edges contacts said cover only when said cover is sealed to said enclosable body.

8. A container system comprising:
a cover including:
a central region;
at least one sidewall extending from said central region;
a first mating interface;
a first sealing interface; and
an enclosable body comprising:
at least one sidewall defining a cavity having an opening;
a second mating interface disposed on said at least one sidewall, said second mating interface engaging said first mating interface;
a second sealing interface disposed on said at least one sidewall, said second mating interface engaging said first sealing interface to form a seal therebetween; and
a sealing indicator disposed within on said at least one sidewall, said sealing indicator configured to indicate that said cover is sealed to said enclosable body.

9. The container system of claim 8, wherein said at least one sidewall of said cover includes at least one inspection element, said at least one inspection element revealing a portion of said sealing indicator when said seal is formed.

10. The container system of claim 8, wherein said central region of said cover includes an aperture.

11. The container system of claim 8, wherein said sealing indicator comprises at least one upper or lower edge, wherein at least one of said upper or lower edges is obscured only when said cover is sealed to said enclosable body.

12. The container system of claim 11, wherein said upper edge of said sealing indicator is obscured only when said cover is sealed to said enclosable body.

13. The container system of claim 11, wherein said lower edge of said sealing indicator is obscured only when said cover is sealed to said enclosable body.

14. The container system of claim 11, wherein at least one of said upper or lower edges is obscured by said cover only when said cover is sealed to said enclosable body.

15. The container system of claim 11, wherein at least one of said upper or lower edges contacts said cover only when said cover is sealed to said enclosable body.

16. The container system of claim 11, wherein a portion of said cover extends over and obscures at least one of said upper or lower edges contacts said cover only when said cover is sealed to said enclosable body.

17. The container system of claim 16, wherein said cover includes a flange extending radially outward from said at least one sidewall of said cover, wherein said flange obscures at least one of said upper or lower edges contacts said cover only when said cover is sealed to said enclosable body.

18. The container system of claim 8, wherein said cover forms a snap-fit connection with said enclosable body.

19. The container system of claim 8, wherein said cover forms a friction-fit connection with said enclosable body.

20. A covering element comprising:
a central region;
at least one sidewall extending from said central region;
a mating and a sealing interface configured to engage with a mating and sealing interface of an enclosable body to form a seal therebetween; and
at least one inspection element disposed in said at least one sidewall, said inspection element configured to reveal a sealing indicator of said enclosable body representing that said cover is sealed to said enclosable body.
